(19) United States
(12) Patent Application Publication
(10) Pub. No.: US 2022/0068065 A1
(43) Pub. Date: Mar. 3, 2022

(54) ACCESS CONTROL INSTALLATION

(71) Applicant: IDEMIA IDENTITY & SECURITY FRANCE, COURBEVOIE (FR)

(72) Inventors: Olivier TOURET, COURBEVOIE (FR); Pierre CHASTEL, COURBEVOIE (FR)

(21) Appl. No.: 17/463,982

(22) Filed: Sep. 1, 2021

(30) Foreign Application Priority Data
Sep. 1, 2020 (FR) 2008883

(51) Int. Cl. G07C 9/25; G06K 9/00; A61B 5/00; A61B 5/0205; A61L 2/10; A61L 2/22; A61L 2/18; A61L 2/26

(52) U.S. Cl. CPC G07C 9/257; A61B 3/112; G06K 9/00255; G06K 9/00288; G06K 9/00013; G06K 9/00604; G06K 9/00617; G06K 9/00087; A61B 5/0077; A61B 5/02055; A61L 2/10; A61L 2/22; A61L 2/18; A61L 2/26; G06K 9/00483

(57) ABSTRACT

An access control installation includes a structure, a moving walkway that extends between two walls of the structure from an entrance to an exit of the installation, a document reader mounted on the structure in the vicinity of the entrance of the installation, a biometric sensor device mounted on the structure at a predetermined distance from the exit of the installation, and an electronic processor unit connected to the document reader and to the biometric sensor device and arranged to compare reference biometric data contained in the document with biometric characteristics supplied by the sensor device in order to verify that they match, the predetermined distance being sufficient, given the speed at which the walkway moves, to allow the electronic processor device to perform the verification operation before the user concerned reaches the exit of the installation.

The present invention relates to the field of identifying and recognizing individuals, e.g. in order to enter into an area with protected access, such as an airport boarding lounge or a border crossing between two countries.

### BACKGROUND OF THE INVENTION

Conventionally, the identity checks for giving access to such areas are performed by operators. The time taken to gain access is then relatively long, particularly during busy periods as can occur in airports during holiday departures.

An automatic device has been devised that comprises:
an airlock;
an integrated circuit (IC) reader at the entrance to the airlock for reading reference biometric data stored in a memory incorporated in a passport; and
a biometric sensor device arranged inside the airlock in order to take an image of a portion of the body of the bearer of the passport and to extract biometric data therefrom for comparison with the reference biometric data.

Nevertheless, although such a device makes it possible to do without an operator, it nevertheless remains necessary to have multiple devices for use during busy periods.

### OBJECT OF THE INVENTION

A particular object of the invention is to improve document verification, in particular verification of identity documents or of access authorizations, and/or to improve biometric verification in areas that are relatively busy.

### SUMMARY OF THE INVENTION

To this end, according to the invention, there is provided an access control installation comprising a structure, a surface that extends between two walls or the structure parallel to the walls to form a floor that is movable relative to the structure and on which users are to stand, and motor means for driving movement of the surface in translation between the walls from an entrance of the installation to an exit of the installation. The installation also comprises at least one item selected. from a document reader and from a biometric sensor device, which item is mounted on the structure at a predetermined distance from the exit of the installation, and an electronic processor unit connected to the document reader and/or to the biometric sensor device and arranged to compare reference data with the data supplied by the document reader and/or by the biometric sensor device in order to verify that they match. The predetermined distance is sufficient, given the speed at which the surface moves, for The electronic processor unit to perform the verification operation before the user concerned reaches the exit of the installation.

Thus, the verification operation is performed while users are moving, which enables verifications to be performed in “hidden” time, while the users are going to their destination, in particular such as an airport boarding lounge or when crossing a border.

According to a particular characteristic of the invention, the biometric sensor device comprises an optical camera for taking images of the faces of users and the electronic processor device is arranged to respond to the images to detect at least one of the following physiological parameters:
user temperature;
user eye brightness;
user pupil dilation;
user eyes open;
sweating; and
user heart rate.

Thus, it is possible to use the camera images not only for biometric purposes, but also to determine the physiological state of a candidate, where the signs in question may be symptoms of anxiety, of illness, of substance abuse, . . .

Advantageously, the electronic processor unit is arranged to evaluate user state of health on the basis of one or more of the physiological parameters and/or is arranged to detect the presence or the absence of masks on the faces of users.

Preferably, the installation includes both a document reader and a biometric sensor device, the document reader being mounted on the structure in the vicinity of the entrance of the installation and the biometric sensor device being mounted on the structure at the predetermined distance from the exit of the installation, and the electronic processor unit is connected to the document reader and to the biometric sensor device and is arranged to compare reference biometric data with the biometric characteristics supplied by the biometric sensor device in order to verify that they match.

Other characteristics and advantages of the invention appear on reading the following description of a particular and nonlimiting embodiment of the invention.

### BRIEF DESCRIPTION OF THE DRAWINGS

Reference is made to the accompanying drawings, in which:

FIG. 1 is a diagrammatic perspective view of an installation of the invention;

FIG. 2 is a diagrammatic end view, partially in section, showing the installation;

FIG. 3 is a diagrammatic view of the installation in longitudinal section on plane III of FIG. 1;

FIG. 4 is a fragmentary diagrammatic view of the end of one of the walls at the exit from the installation;

FIG. 5 is a fragmentary diagrammatic view from above of one of the handrails; and

FIG. 6 is a fragmentary diagrammatic view of the handrail in a variant embodiment, shown in cross-section on line VI-VI of FIG. 5.

### DETAILED DESCRIPTION OF THE INVENTION

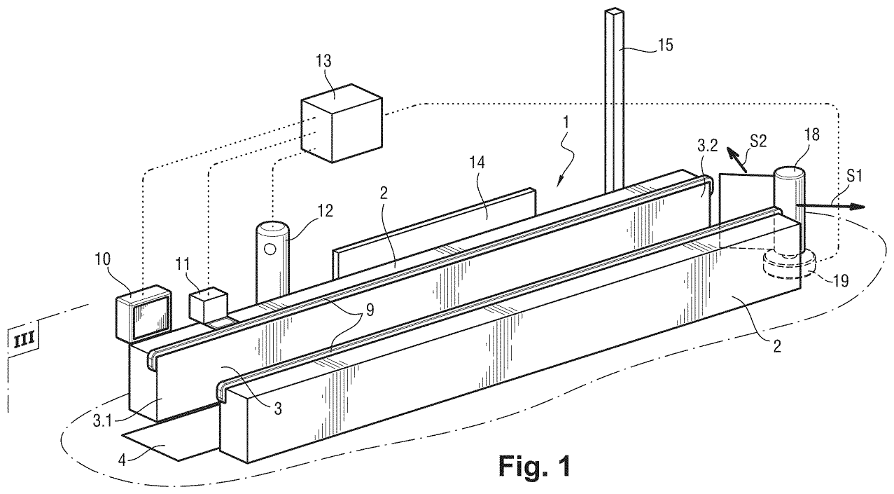

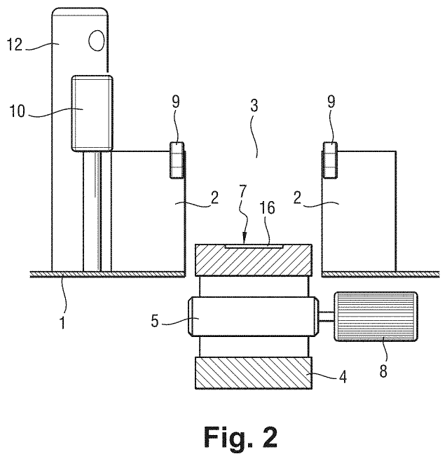

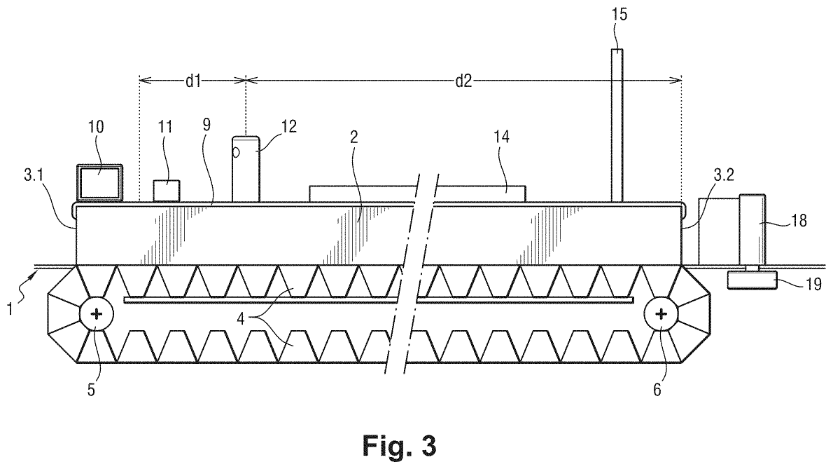

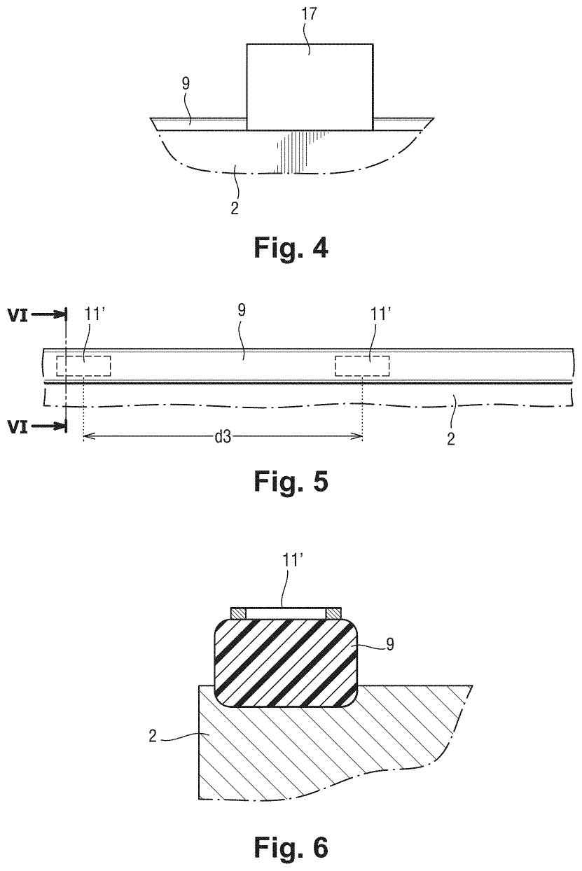

With reference to FIGS. 1 to 5, the access control installation of the invention comprises a structure given overall reference 1 and that has two walls 2 defining between them a passage 3 having an entrance 3.1 and an exit 3.2.

In the passage 3, and along the two walls 2, there extends a moving walkway 4 that is movable relative to the structure 1 in parallel with the walls 2. The moving walkway 4 is a chain. made up of links that are hinged to one another and that pass around two pulleys 5 and 6 so as to form an elongate loop having a top strand and a bottom strand. While the links are in the top strand of the loop, they make up a top surface that forms a floor 7 for users to stand on. The pullet 6 is driven in rotation by a motor 8 to move each of the links in the top strand away from the entrance 3.1 towards the exit 3.2 (and naturally to move the links in the bottom strand in the opposite direction). The moving walkway 4 could be made in some other way, e.g. by means of a conveyor belt.

A handrail 9 is mounted on each wall 2. Each handrail 9 forms an elongate loop around pulleys (not shown) having a top strand that extends on the top surface of the wall 2 and a bottom strand that runs inside of the wall 2. One of the pulleys is motor-driven so as to drive the handrail 9 synchronously with the moving walkway 4.

The motors of the handrail 9 and of the moving walkway 4 are organized to cause users of the installation to move at a speed V. By way of example, the speed V may lie the range 0.5 meters per second (m/s) to 1 m/s.

The installation. also comprises a contactless document reader 10, a fingerprint sensor 11, and a face-sensor camera 12, all three of which are connected to an electronic processor unit 13.

The reader 10 is arranged to read a memory incorporated in a document. In this example, the memory is an integrated circuit connected to an antenna. The reader 10 is itself known and comprises a processor connected to an antenna and programmed to interrogate the integrated circuit so as to obtain data that it contains. Specifically, the document is a passport and the integrated circuit contains biometric data of the bearer of the passport, specifically fingerprint biometric data and face biometric data. The reader 10 is mounted on one of the walls 2 in the vicinity of the entrance 3.1 of the passage 3.

In this example, the fingerprint sensor 11 is an optical sensor having a field of view covering a sensing zone in which the user passes a hand so as to allow the sensor to take at least one image of the dermatoglyphs of one or more of the user's fingers. The sensor presents acquisition speed and resolution that enable images of moving hands to be acquired with sufficient detail to be able to extract fingerprint biometric characteristics. The fingerprint reader 11 is mounted on one of the walls 2 in the vicinity of the entrance 3.1 of the passage 3.

The face sensor camera 12 has a sensor that presents acquisition speed and resolution that enable images of moving faces to be acquired with sufficient detail to be able to extract face biometric characteristics. In this example, the face sensor camera 12 is mounted on a post having its base fastened to the structure 1 beside one of the walls 2. The face sensor camera 12 has a field of view covering a depth d1 along the passage 3, which depth is sufficient to enable the face sensor camera 12 to take at least one image of a user's face, given the speed V at which the user is moving along the passage 3.

The electronic processor unit 13 comprises a processor and memories containing biometric recognition programs arranged to compare the reference biometric data contained in the documents with the biometric characteristics as supplied by the fingerprint sensor 11 and by the face sensor camera 12, in order to verify whether they match. Such biometric recognition (or “matching”) programs are themselves known.

The fingerprint sensor 11 and the face sensor camera 12 are placed along the passage 3 at a distance d2 from the ext. 3.2 of the passage 3. This distance d2 is sufficient, given. the speed V at which. the walkway moves, for the electronic processor unit 13 to perform the biometric verification. operation before the user concerned reaches the exit 3.2 of the passage 3.

In this example, the installation also comprises a barrier 18 arranged. outside the passage 3 in. the vicinity of the exit 3.2 of the installation and connected to a motor 19 so as to be movable between two positions so as to direct users either in a direction S1 or else in a direction S2. The motor 19 is connected to the processor unit 13, which causes the barrier to be moved towards one or the other of its two positions, depending on the result of the biometric verification. For example, when the biometric verification. is positive, the user is directed in the direction S1 towards a direct exit without further checks, whereas when the biometric verification is negative, the user is directed in the direction S2 towards an operator who carries out additional checks.

Preferably, users are spaced apart by about 50 centimeters (cm) in order to facilitate the sensing operations and in order to distinguish clearly between users.

Advantageously, the electronic processor unit 13 also executes a program for verifying that the biometric characteristics are compatible with personal information about the user that is stored in the memory of the passport, such as age, gender, eye color, . . .

Preferably, the electronic processor unit 13 is arranged to respond to the images supplied by the face sensor camera 12 by detecting at least one of the following physiological parameters:
user temperature;
user eye brightness;
user pupil dilation;
user eyes open;
sweating; and
user heart rate.

The electronic processor unit 13 is arranged to evaluate user state of health on the basis of one or more of the physiological parameters. Preferably, the electronic unit makes use of a neural network to respond to the physiological parameters in order to classify users as users who are presumed to be in good health and users who are presumed to be in poor health. Advantageously, provision is made to take account of ambient temperature while performing this evaluation.

The electronic processor unit 13 is also arranged to detect the presence of a protective mask (of the surgical mask type covering both the mouth and the nose) on the face of a user. The facial recognition algorithm is preferably configured to make biometric recognition possible in spite of the presence of such a protective mask (by giving precedence to biometric characteristics that are left uncovered by a mask).

The installation also comprises some additional sensors, namely:
a uniqueness sensor comprising a camera, a horizontal line of lighting 14, and a vertical line of lighting 15 so as to be able to distinguish between two users who are pressed close together (one behind the other, one on the other, one beside the other, . . . ). It should be observed that the longer the time it takes to travel along the passage 3, the more difficult it becomes for two users to remain pressed close together without that being detected; and
a weight sensor 17 mounted on each of the links making up the moving walkway 4. The weight measurement can serve co reveal that two users are standing on the same link, pressed close against each other.

As shown in FIG. 5, the installation includes a disinfection tunnel 17 mounted on each wall 2, with the handrail 9 passing therethrough. By way of example, the disinfection tunnel 17 may include both an ultraviolet (UV) lamp for projecting UV radiation onto the top surface of the handrail 9 and also a spray, for spraying a disinfectant solution onto the top surface of the handrail 9.

In a variant, fingerprint sensors 11′ may be placed directly on one of the handrails. The fingerprint sensors 11′ are sensors of capacitive or thin-film or thin-film transistor (TFT) type. The locations of the fingerprint sensors 11′ are made visible on the top surface of the handrail 9 so that users know where to place their hands in order to enable their fingerprints to be sensed. The fingerprint sensors 11′ are spaced apart from one another by a distance d3 corresponding to the spacing that is desired between. users on the moving walkway 4.

Naturally, the invention is not limited to the embodiment described and covers any variant coming within the ambit of the invention as defined by the claims.

In particular, the installation may be of structure other than that described, and by way of example it may be in the form of a moving walkway on a slope or of an escalator.

The memory incorporated in the document may be in the form of an integrated circuit, of a magnetic strip, of a barcode, of an image, . . . . The document reader may be an optical camera having a field in which the barcode or the image is to be presented.

The installation may include at least one of the following biometric sensor devices:
a fingerprint sensor;
an iris sensor; and
a face sensor.

The installation may optionally include at least one additional sensor connected to the processor unit and selected from:
a weight sensor arranged on the moving surface;
a user height sensor;
a millimeter wave camera or a terahertz camera;
a user temperature sensor;
a detector of explosives;
a uniqueness detector by providing horizontal and vertical lighting along the walkway;
a boarding card reader at the entrance to the installation; . . .

The invention can be used with passports in which the memory contains biometric data for the face only. Although not essential, it nevertheless remains advantageous, in spite of that, to sense fingerprints, e.g. in order to be able to use the fingerprints to search in databases of wanted persons or of missing persons, in order to check visas, . . . . This may likewise apply to other types of biometric measurement.

The only constraint on the positions of the biometric sensors is that they must be situated far enough away from the exit to enable the appropriate checks to be performed. before the user reaches the exit.

There may be a plurality of sensor cameras (e.g. in order to improve accuracy), in particular on the right and on the left of the moving surface.

Biometric sensors that are suitable for sensing biometric data remotely and in motion are particularly appropriate for the invention (e.g. face sensors and iris sensors).

The document reader may comprise an optical reader member for performing a stage of reading the document optically, together with members distributed all along the handrail in order to perform contactless reading of the integrated circuit in the document. The IC may thus be read in parallel while acquiring biometric data.

The reference biometric data may be contained either in the identity document or else in a database hosted in a server to which the device is connected. The database is preferably searched on the basis of information contained in the document (document number, name of bearer, . . . ).

The installation need not include a document reader. Under all circumstances, the document reader (s) and the biometric sensor device (s) are positioned along the structure at distances that are sufficient, given the speed V at which the moving surface 7 moves, to enable the electronic processor unit 13 to perform the verification operations before the user concerned reaches the exit 3.2 of the installation.

Provision may be made to enable the processor unit 13 to control the motor 8 so as to slow down the moving walkway 4 whenever biometric verification of a user is taking longer than. expected, so that the biometric verification can be completed by the time the user reaches the exit from the passage 3.

The barrier is optional. By way of example, switching may be done by an operator or by a sign.

1. An access control installation comprising a structure, a surface that extends between two walls of the structure to form a floor that is movable relative to the structure parallel to the walls and on which users are to stand, and motor means for driving movement of the surface in translation between the walls from an entrance of the installation to an exit of the installation; and at least one item selected from a document reader and from a biometric sensor device, which item is mounted on the structure at a predetermined distance from the exit of the installation, and an electronic processor unit connected to the document reader and/or to the biometric sensor device and arranged to compare reference data with the data supplied by the document reader and/or by the biometric sensor device in order to verify that they match, the predetermined distance being sufficient, given the speed at which the surface moves, for the electronic processor unit to perform the verification operation before the user concerned reaches the exit of the installation.

2. The installation according to claim 1, wherein the biometric sensor device comprising an optical camera for taking images of the faces of users.

3. The installation according to claim 2, wherein the electronic processor device is arranged to respond to the images to detect at least one of the following physiological parameters:
user temperature;
user eye brightness;
user pupil dilation;
user eyes open;
sweating; and
user heart rate.

4. The installation according to claim 3, wherein the electronic processor unit is arranged to evaluate user state of health on the basis of one or more of the physiological parameters.

5. The installation according to claim 2, wherein the electronic processor unit is arranged to detect the presence or the absence of masks on the faces of users.

6. The installation according to claim 1, including both a document reader and a biometric sensor device, the document reader being mounted on the structure in the vicinity of the entrance of the installation and the biometric sensor device being mounted on the structure at the predetermined distance from the exit of the installation, and the electronic processor unit is connected to the document reader and to the biometric sensor device and is arranged to compare reference biometric data with the biometric characteristics supplied by the biometric sensor device in order to verify that they match.

7. The installation according to claim 1, including at least one of the following biometric sensor devices:
a fingerprint sensor;
an iris sensor; and
a face sensor.

8. The installation according to claim 1, including at least one additional sensor connected to the processor unit and selected from:
a weight sensor arranged on the moving surface;
a user height sensor;
a millimeter wave camera or a terahertz camera; and
a user temperature sensor.

9. The installation according to claim 1, including a uniqueness detector serving to detect two users pressed close against each other.

10. The installation according to claim 1, including handrails running along the top surfaces of the walls and moving synchronously with the moving surface.

11. The installation according to claim 10, including a disinfection member for disinfecting each handrail.

12. The installation according to claim 10, comprising a plurality of biometric sensor devices each including a fingerprint sensor, the fingerprint sensors being fastened to one of the handrails in mutually spaced-apart manner.

13. The installation according to claim 1, including a barrier arranged at the exit of the installation and connected to a motor controlled by the processor unit so as to put the barrier in two distinct positions as a function of the results of the verification.

14. The installation according to claim 1, wherein the electronic processor unit is arranged to verify that the extracted biometric characteristics are compatible with information relating to the user and appearing in the document.
